(19) United States
(12) Patent Application Publication
(10) Pub. No.: US 2014/0212484 A1
(43) Pub. Date: Jul. 31, 2014

(54) MULTILAYERED POLYELECTROLYTE-BASED CAPSULES FOR CELL ENCAPSULATION AND DELIVERY OF THERAPEUTIC COMPOSITIONS

(71) Applicant: Islet Sciences, Inc., La Jolla, CA (US)

(72) Inventors: Jain KROTZ, San Diego, CA (US); Amish PATEL, San Diego, CA (US)

(73) Assignee: Islet Sciences, Inc., La Jolla, CA (US)

(21) Appl. No.: 13/968,577

(22) Filed: Aug. 16, 2013

Related U.S. Application Data
(63) Continuation of application No. 11/868,205, filed on Oct. 5, 2007, now abandoned.
(60) Provisional application No. 60/828,503, filed on Oct. 6, 2006.

(51) Int. Cl. A61K 9/48; A61K 45/06; A61K 35/39

(52) U.S. Cl. CPC A61K 9/4891; A61K 9/4808; A61K 35/39; A61K 45/06
USPC 424/452; 424/93.7; 424/158.1

(57) ABSTRACT

The present invention provides novel, biocompatible matrices for cell encapsulation and transplantation. It further provides methods for delivering agents to encapsulated cells and to the local environment of a host system. The invention also provides methods for targeting and manipulating particular cells and/or proteins of the host system. In a composition aspect of the invention, a composition including a collection of capsules is provided. The capsules comprise an inner core, and the inner core is covered by an outer shell composed of a positive polyelectrolyte and a negative polyelectrolyte. The inner core of the capsules contains at least one cell.

### CROSS-REFERENCE TO RELATED APPLICATION

This application claims priority to U.S. Provisional Patent Application Ser. No. 60/828,503, entitled “Multilayered Polyelectrolyte-Based Capsules For Cell Encapsulation And Delivery Of Therapeutic Compositions” and filed on Oct. 6, 2006, the entire disclosure of which is hereby incorporated by reference in its entirety.

### FIELD OF THE INVENTION

The present invention provides novel, biocompatible matrices for cell encapsulation and transplantation. It further provides methods for delivering agents to encapsulated cells and to the local environment of a host system. The invention also provides methods for targeting and manipulating particular cells and/or proteins of the host system.

### BACKGROUND OF THE INVENTION

Treating human diseases arising from hormone or protein deficiencies using cells is currently a limited methodology, since the cells are often destroyed by a recipient's immune system. One may attempt to minimize such immune rejections through the administration of immuno-suppressive drugs. The drugs, however, have multiple drawbacks and oftentimes exhibit detrimental side effects. Another strategy to reduce rejection involves the encapsulation of the cells in a biocompatible matrix (i.e., immunoisolation).

Immunoisolation of cells not only helps to prevent rejection of human cells, it also provides one with an opportunity to administer cells from non-human species (i.e., xenografts). This opportunity is especially important in the case of Type 1 diabetes, where demand for transplantable human islet cells far exceeds the supply of donor pancreases. Inclusion of non-human islet cells, such as porcine islet cells, in the supply chain would substantially address current and future demand.

The success of xenografts depends upon multiple factors including the capsule's ability to protect the encapsulated cells from immune reactions and the capsule's ability to allow the transport of nutrients to the cells. Accordingly, ideal capsules, or encapsulation matrix, would be designed to limit attacks from immune cells and cytokines derived from macrophages (e.g., IL-1β and TNF-α) without preventing the diffusion of materials necessary for cell viability.

A number of encapsulation methods and polymeric materials have been used for cells, including Alginate. For example, Alginate-polylysine capsule formation is reported in U.S. Pat. No. 4,391,909; an alginate-chitosan technique is reported in U.S. Pat. No. 4,744,933; and a polyacrylate encapsulation technique is reported in U.S. Pat. No. 4,353,888.

Despite the volume of work in the field of cell encapsulation, there still is not a capsule formulation that can provide long-term biocompatibility and immuno-protection for encapsulated cells.

### SUMMARY OF THE INVENTION

The present invention provides novel, biocompatible matrices for cell encapsulation and transplantation. It further provides methods for delivering agents to encapsulated cells and to the local environment of a host system. The invention also provides methods for targeting and manipulating particular cells and/or proteins of the host system.

In a composition aspect, a composition including a plurality of capsules is provided. In some embodiments, the capsules have an inner core, and the inner core is enclosed by an outer shell including a positive polyelectrolyte and a negative polyelectrolyte. In some embodiments, the inner core of the capsules contains at least one cell. In some embodiments, the composition includes a plurality of capsules, wherein each capsule comprises an inner core comprising at least one cell; and an outer shell comprising a positive polyelectrolyte and a negative polyelectrolyte; and wherein the outer shell encloses the inner core. In some embodiments, the inner core comprises alginate. In some embodiments, the positive polyelectrolyte is one or more of chitosan, protamine sulfate, polybrene, poly(L-lysine), poly(allylamine hydrochloride), poly(ethylene imine) or poly(ethylene glycol-co-dimethylaminoethyl methacrylate). In some embodiments, the negative polyelectrolyte is one or more of: poly(styrene sulfate), polyacrylamideomethyl propane sulfonic acid, poly(lactic acid), cellulose sulfate, alginate, hyaluronic acid, chondroitin sulfate or poly(ethylene glycol-co-methacrylic acid). In some embodiments, the outer shell is formed by the molecular assembly of oppositely charged polymers in a layer by layer manner. In some embodiments a positive polyelectrolyte disposed between the inner core and a negative polyelectrolyte in the outer shell. In some embodiments a negative polyelectrolyte is disposed between the inner core and a positive polyelectrolyte in the outer shell. In some embodiments the polyelectrolyte disposed in the outermost portion of the outer shell comprises a negative polyelectrolyte modified with a protein and polyethylene glycol. In some embodiments the polyelectrolyte disposed in the outermost portion of the outer shell comprises a negative polyelectrolyte modified with at least one anti-cytokine antibody or at least one RGD motif. In some embodiments the capsules exhibit a porosity control equal to the diffusional restriction of dextrans of defined molecular weight, and wherein the diffusional restriction is controlled in the range of about twenty percent molecular weight cutoff to about ninety percent molecular weight cutoff for a 10 kD dextran. In some embodiments, the capsules exhibit a porosity control equal to the diffusional restriction of dextrans of defined molecular weight, and wherein the diffusional restriction is controlled in the range of about thirty percent molecular weight cutoff to about eighty percent molecular weight cutoff for a 10 kD dextran. In some embodiments, the capsules exhibit a porosity control equal to the diffusional restriction of dextrans of defined molecular weight, and wherein the diffusional restriction is controlled in the range of about thirty percent molecular weight cutoff to about ninety percent molecular weight cutoff for a 40 kD dextran. In some embodiments the capsule comprises an effective pore size of less than about 10 nm. In some embodiments the composition includes at least one anti-inflammatory drug conjugated to the outer shell. In some embodiments the composition includes anti-apoptotic agents in the inner core. In some embodiments the composition includes at least one immuno-suppressive drug conjugated to the outer shell. In some embodiments the composition includes at least one targeting-type molecule conjugated to the outer shell.

In a method aspect of the present invention, a method of treating a disease in a patient is provided. In some embodiments, the method includes administering one or more embodiments of a composition of the present invention to a patient. In some embodiments, the composition is administered through intraperitoneal injection. In some embodiments the disease treated is diabetes. In some embodiments the composition administered includes at least one pancreatic islet cell. In some embodiments the cells in the composition exhibit a viability of greater than about 80 percent within 24 hours after administration. In some embodiments the cells in the composition exhibit a viability of greater than about 80 percent after ninety-six hours of administration.

In another method aspect of the present invention, a method of making a capsule including a therapeutic composition is provided. In some embodiments, the method comprises the steps of: (a) forming a suspension of a first capsule in an aqueous solution, wherein the first capsule has a base membranous structure, and wherein the base membranous structure comprises alginate, and wherein the first capsule comprises a therapeutic composition; (b) adding a first polyelectrolyte to the suspension to form a first polyelectrolyte-coated capsule; and, (c) adding a second polyelectrolyte to the first polyelectrolyte-coated capsule, thereby forming the capsule comprising a therapeutic composition. In some embodiments the method includes the steps of forming an inner core encapsulating cells by forming a suspension of a first capsule in an aqueous solution, wherein the inner core comprises alginate; forming an outer shell of the capsule by adding a first polyelectrolyte to the suspension to form a first polyelectrolyte-coated capsule; and adding a second polyelectrolyte to the first polyelectrolyte-coated capsule. In some embodiments, the method also includes the step of conjugating, the outer shell of the first capsule to at least one anti-inflammatory drug. In some embodiments, the method also includes the step of modifying the inner core with a cell adhesive protein moiety. In some embodiments the method also includes the step of adding anti-apoptotic agents that are encapsulated in the inner core. In some embodiments, the first polyelectrolyte is a positively charged polyelectrolyte that is chitosan, protamine sulfate, polybrene, poly(L-lysine), poly(allylamine hydrochloride), poly(ethylene imine) or poly(ethylene glycol-co-dimethylaminoethyl methacrylate). In some embodiments, the first polyelectrolyte is a negatively charged polyelectrolyte selected from a group consisting of poly(styrene sulfate), polyacrylamideomethyl propane sulfonic acid, poly(lactic acid), cellulose sulfate, alginate, hyaluronic acid, chondroitin sulfate or poly(ethylene glycol-co-methacrylic acid).

### BRIEF DESCRIPTION OF THE FIGURES

FIG. 1 is a schematic of the formation of molecularly assembled outer shell around islet encapsulated alginate beads.

FIG. 2A is a Scanning Electron Micrograph of Alginate beads and FIG. 2B is a Scanning Electron Micrograph of Alginate beads after the outer shell has been formed.

FIGS. 3A and 3B are Confocal microscopic images showing the penetration into alginate beads by a FITC labeled polyelectrolyte of MW 150 kD.

FIG. 4 is a graph illustrating Percentage reduction in diffusion for 10 kDa and 40 kDa dextrans compared to alginate upon formation of an outer shell composed of (PolyAllylamine/Polystyrene sulfonic acid)3 [PAH/PSS].

FIG. 5 is a graph illustrating the percentage reduction in permeability (% cut off) compared to alginate upon formation of an outer shell composed of (Chitosan/Human serum albumin)₃ [Chi/HuSA].

FIG. 6 is a graph illustrating the percentage reduction in permeability (% cut off) compared to alginate upon formation of an outer shell composed of (Chitosan/Polystyrene sulfonic acid)₁₋₃ (Chi/PSS).

FIG. 7 is a graph illustrating percentage reduction in permeability compared to alginate upon formation of an outer shell composed of (Chitosan/Polystyrene sulfonic acid)₁₋₃ |Chi/PSS|.

FIGS. 8A-C illustrate solubility changes induced by outer shell formation in a calcium chelating medium: (A) shows the core in the presence of EDTA; (B) shows subsequent incubation of these EDTA treated beads in a calcium containing buffer leads to re-gelation of the alginate inner core (C).

FIG. 9 is a graph showing capsule stability as a function of number of bilayers and temperature for 20 kD dextran.

FIG. 10 is a graph showing capsule stability as a function of number of bilayers and temperature for 70 kD dextran.

FIG. 11 is a graph showing capsule stability as a function of time in culture medium at 25° C. for a 3 bilayer system.

FIG. 12 is a graph showing capsule stability as a function of time in culture medium at 25° C. for a 6 bilayer system.

FIGS. 13A-J are confocal images of fluorescently labeled capsules transplanted for 3 weeks in vivo.

FIG. 14 is a graph showing the comparison of oxygen consumption rates of LbL formulations and alginate control beads.

FIG. 15 is a graph showing graft function in mice of an LbL formulation and its control-alginate.

### DETAILED DESCRIPTION OF THE INVENTION

In the following paragraphs, the present invention will be described in detail by way of example with reference to the attached figures. Throughout this description, the embodiments and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must comprise the referenced feature(s).

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary illustrations and figures. As will become apparent to one of ordinary skill in the art after reading this document, the illustrated embodiments and their various alternatives may be implemented without confinement to the illustrated examples.

The present invention provides novel, biocompatible matrices for cell encapsulation and transplantation. It further provides methods for delivering agents to encapsulated cells and to the local environment of a host system. The invention also provides methods for targeting and manipulating particular cells and/or proteins of the host system.

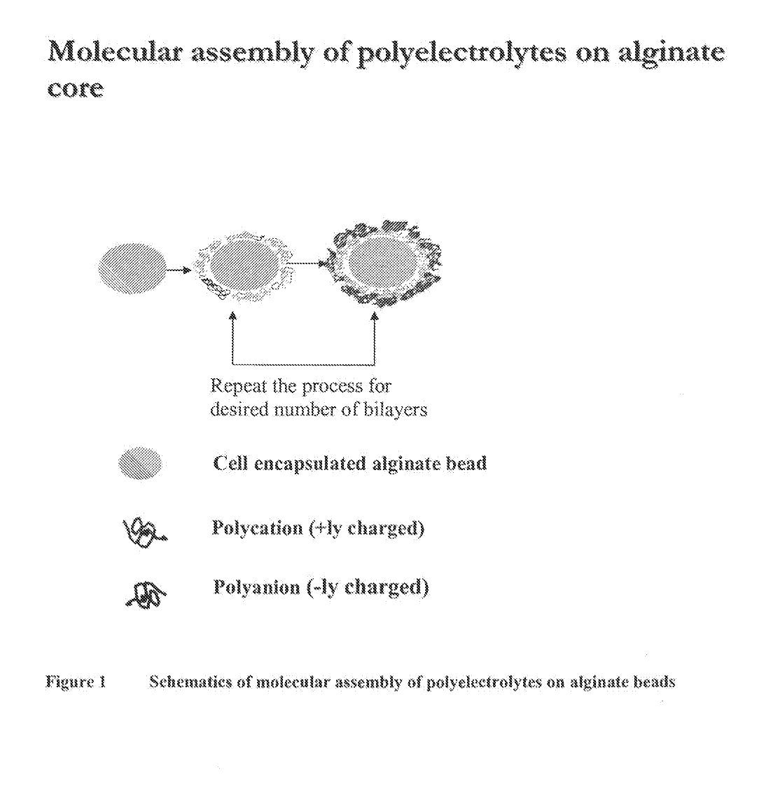

The capsules comprise an inner, polymer-based core and an outer shell composed of multiple layers of oppositely charged polyelectrolytes. In one embodiment, the outer shell is assembled via ionic interactions. In some embodiments, the outer shell is formed by sequential deposition of oppositely charged polyelectrolytes in a controlled LbL process (FIG. 1). In some embodiments, the process is performed in aqueous media under ambient conditions and is therefore compatible with sensitive systems such as encapsulated cells and enzymes.

The inner core may comprise any suitable polymer. Typically, however, the structure comprises alginate in either a natural or modified form. Modified alginates comprise polymers having an alginate backbone to which another, non-alginate polymer is grafted. The grafted polymer contains one or more types of monomers, which are capable of undergoing a radical addition and are typically of the following structure: CH₂═C(R¹)EWG, where R¹ is selected from a group of moieties consisting of hydrogen, C1 to C6 alkyl and aryl, and where “EWG” is an electron withdrawing group. Nonlimiting examples of electron withdrawing groups include: —C(O)R², where R² is hydrogen, alkyl, substituted alkyl, aryl, or substituted aryl; —C(O)OR², —C(O)NR²R³, where R³ is hydrogen, alkyl, substituted alkyl, aryl, or substituted aryl; —C(O)OR²; —C(O)NR²R³, where R³ is hydrogen, alkyl, substituted alkyl, aryl or substituted aryl; —S(O)₂OR²; —S(O)₂NR²R³; and, —P(O)₂OR². R¹ is preferably hydrogen or methyl. The inner core can also be a blend of alginate and another water soluble, biocompatible polyelectrolyte, such as chitosan, albumin, chondroitin sulfate and hyaluronic acid.

The polyelectrolyte deposited on the inner core structure can be either the positively charged polyelectrolyte or the negatively charged polyelectrolyte. Polyelectrolytes may be layered such that combinations of opposite charge are produced—e.g., positive polyelectrolyte then negative polyelectrolyte then positive polyelectrolyte.

In some embodiments, the positive polyelectrolytes of the outer shell may comprise polymers such as chitosan, poly(allylamine hydrochloride) (i.e., “PAH”), poly(ethylene glycol-co-dimethylaminoethyl methacrylate), hexamethridine dibromide (i.e., “HDM”), protamine sulfate (i.e., “ProtS”), chemically modified versions of the preceding polymers, and mixtures thereof.

In some embodiments, the negative polyelectrolytes of the outer shell may comprise polymers such as poly(styrene sulfonate) (i.e., “PSS”), albumin, hyaluronic acid, chondroitin sulfate, polyacrylamido methylpropane sulfonic acid (i.e., “polyAMPS”), polyethyleneglycol-co-methacrylic acid, cellulose, sulfate, alginate, modified versions of the preceding polymers, and mixtures thereof. Typically, the negative polyelectrolytes used are poly(styrene sulfonate), a chemically-modified poly(styrene sulfonate) derivative, polyAMPS, and PEG copolymers.

In some embodiments, the polyelectrolyte polymers discussed in the preceding paragraphs may be chemically-modified in a variety of ways. For instance, the polymers may be pegylated through the use of an appropriate spacer, and/or one or more molecules that target specific tissue may be attached, and/or one or more anti-inflammatory or immuno-suppressive drugs may be attached. Typical targeting-type molecules include anti-cytokine antibodies, and polypeptide sequences such as those comprising an RGD motif or other integrin-based polypeptides. Such polypeptides, comprising an RGD or like motif, are known to facilitate the attachment of capsules to blood vessels.

Anti-inflammatory drugs are either steroidal or non-steroidal. Such drugs include, without limitation, the following: aspirin, salisilate, diflunisal; ibuprofen, ketoprofen, nabumetone, piroxicam, naproxen, diclofenac, indomethacin, sulindac, tolmetin, etodolac, ketorolac, oxaprozin, celecoxib, prednisone, prednisolone, acetaminophen, buprenorphine, butorphanol, codeine, dextropropoxyphene, dihydrocodeine, fentanyl, hydrocodone, hydromorphone, ketobemidone, nalbuphine, oxycodone, oxymorphone, pentazocine, pethidine, tramadol, acetylsalicylic acid, ethenzamide, aminophenazone, metamizole, phenazone, phenacetin, ziconotide, tetrahydrocannabinol, choline salicylate, magnesium salicylate, sodium salicylate.

Immuno-suppressive drugs include, without limitation, the following: glucocorticoids, cytostatics, drugs acting on immunophilins and, specifically, cyclosporine; tacrolimus; Deoxyspergualin (DSG) and sirolimus.

Anti-cytokine antibodies may either be polyclonal or monoclonal, although monoclonal are preferred. Nonlimiting examples of such antibodies include: anti-IL-1α antibodies; anti-IL-10 antibodies; anti-GM-CSF antibodies; anti-IL-1β antibodies; anti-IL-12 antibodies; anti-IP-10 antibodies; anti-IL-3 antibodies; anti-IL-4 antibodies; anti-TNFα antibodies; anti-MIP1α antibodies; anti-IL-6 antibodies; anti-MIP1β antibodies; anti-leptin antibodies; anti-IL-8 antibodies; and, anti MIP-5 antibodies.

RGD motifs comprise at least the three amino acid sequence Arginine-Glycine-Aspartate. The motifs may be either linear or circular, and the three amino acid sequence is typically included in a polypeptide that is at least 5 amino acids in length. In certain cases, the polypeptide is at least 10 or 15 amino acids in length.

The modified polyelectrolytes may be synthesized through any suitable method. A typical method involves the acylation of free amino residues on the polymer (e.g., chitosan) using the Maillard reaction. Esterification of free alcohol residues is another common method for modification.

As noted above, the capsule membrane comprises at least one positively charged polyelectrolyte and at least one negatively charged polyelectrolyte. This combination of oppositely charged polyelectrolytes is referred to as a “bilayer.” Embodiments may have any suitable number of bilayers. For example, embodiments may have a single bilayer, two bilayers, three bilayers, four bilayers, five bilayers, or even more than five bilayers.

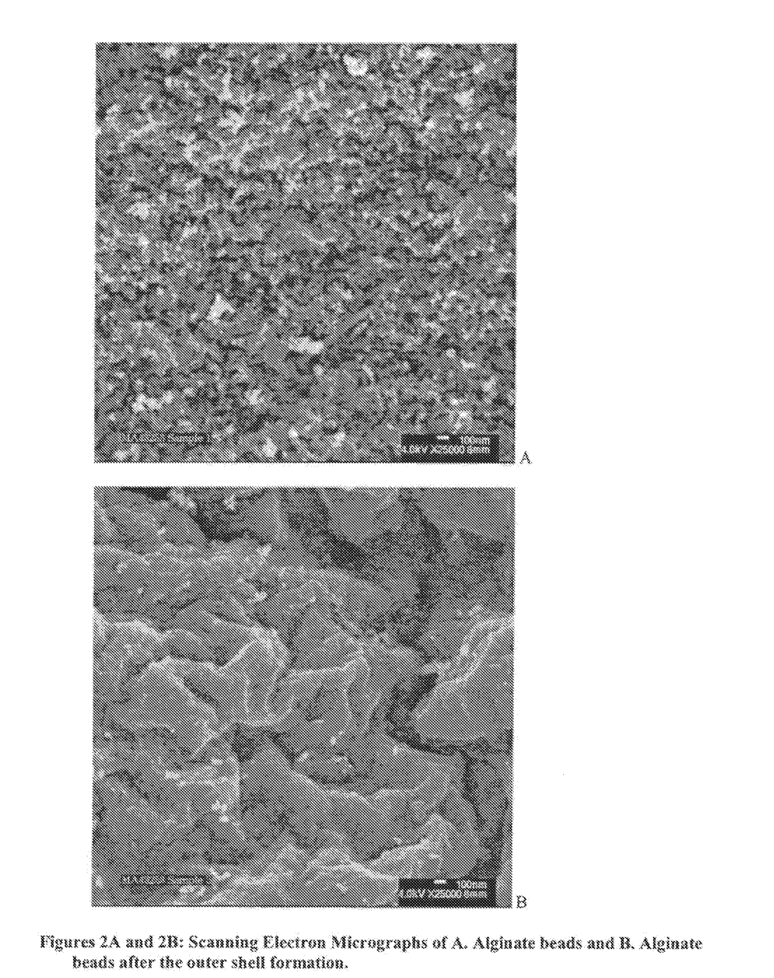

Capsule formation by layering of polyelectrolytes around a core alginate bead allows one to more easily control the porosity, permeability and/or morphology of a microparticle. Morphological changes as measured by scanning electron microscope of alginate beads and capsules formed by layering of polyelectrolytes on alginate are shown in FIG. 2.

As shown in FIG. 2A, alginate beads can have a wide range of pore dimensions on their surface. For instance, pores in the range of 10 to 100 nm are clear from the high resolution image (i.e., at 25,000×). Creation of an outer shell through the molecular assembly of polyelectrolytes such as chitosan and polystyrene sulfonic acid significantly reduces porosity (see FIG. 2B).

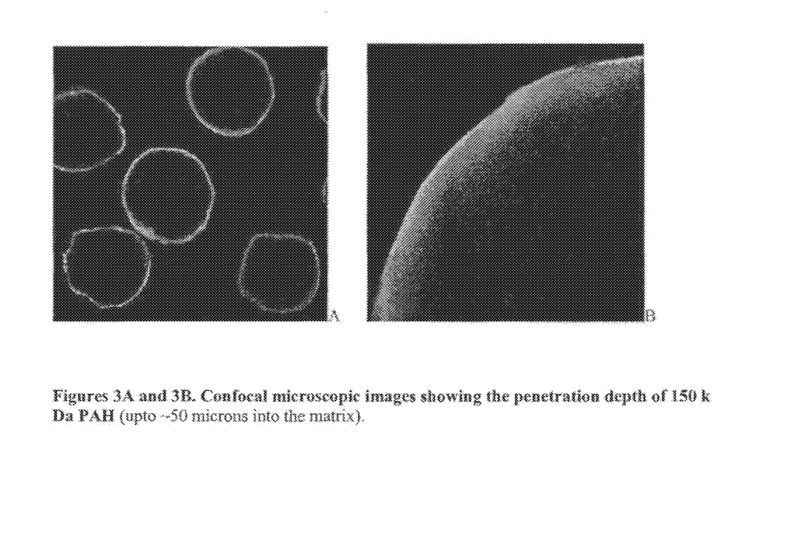

One can vary the thickness of the outer shell by varying the number of polyelectrolyte layers deposited. Layering the alginate inner core with a fluorophore-labeled polycation (e.g., polyallylamine, “PAA”, MW 150 kD) and subsequently evaluating fluorescence intensity on the beads (confocal microscopy) showed the polycation can penetrate up to a depth of ˜50 micron into the inner core matrix (FIG. 3). Penetration depth of the outer layers can be increased by choosing polyelectrolytes of lower molecular weight.

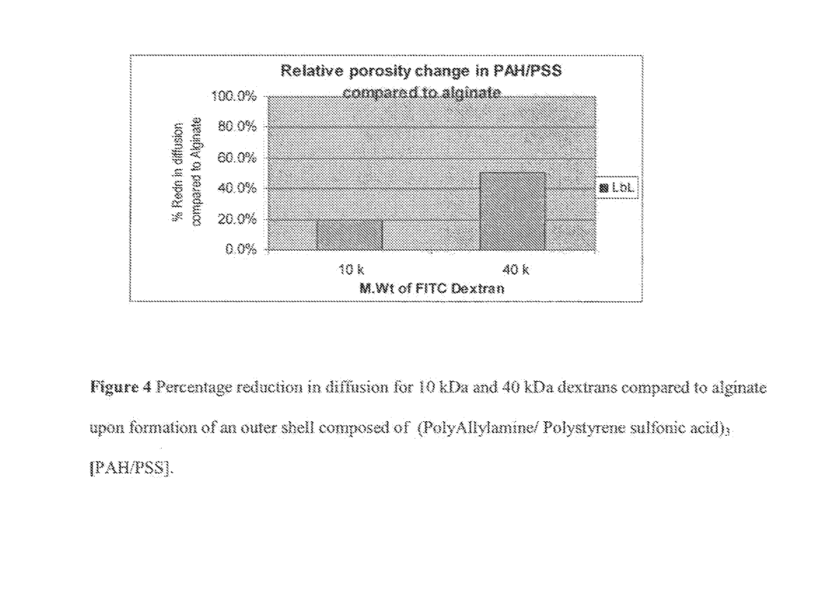

One can measure the permeability (i.e., porosity) difference of alginate beads before and after outer shell (i.e., capsule) formation by monitoring the diffusion of a well characterized molecule such as Dextran. The diffusion of fluorophore-labeled (i.e., FITC) dextran of different molecular weights is first quantified and compared with respect to different formulations. Typical results obtained from a PAA/PSS capsule system are shown in FIG. 4.

For example, in embodiments where the outer shell is formed by a combination of PAA (polycation) and PSS (polyanion), there is an about 20% reduction in the permeability of a 10 kDa dextran and an about 50% reduction in the permeability of a 40 kDa dextran, as compared to alginate beads. Such reduction in molecular diffusion is indicative of reduced capsule permeability. The relative reduction in permeability is correlated to molecular weight cut off (MWCO), which may be important for the long term functioning of encapsulated cells in vivo.

The process of forming the outer shell via molecular assembly is a controlled process and can be optimized to induce the level of diffusional restrictions needed for a particular application. The diffusional restriction (or MWCO) can be controlled by:

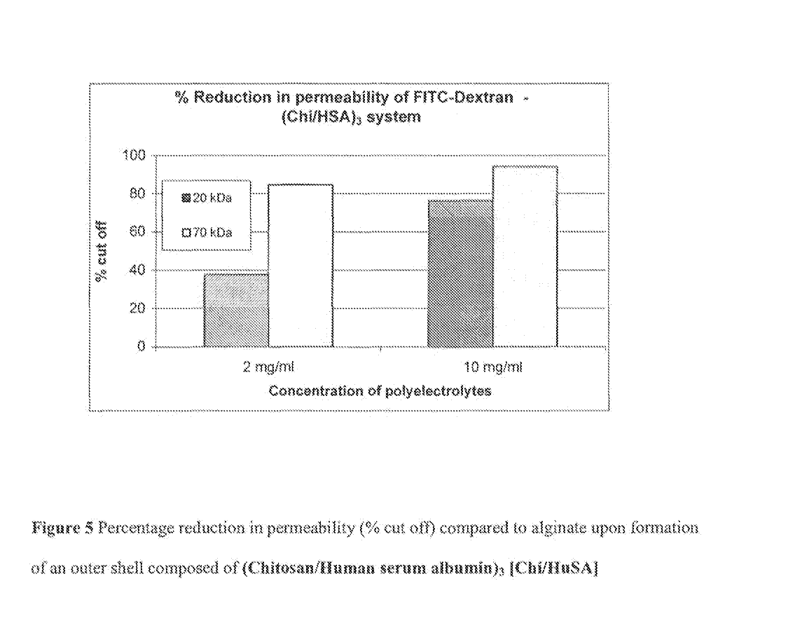

Varying the concentration of polyelectrolytes—an example of this system is shown in FIG. 5 where Chitosan and Human serum albumin are used as the polyelectrolytes at two different concentrations, viz. 3 mg/ml and 10 mg/ml. At 3 mg/ml concentration the formed outer shell imparts a diffusional restriction of about 35% for 10 kDa Dextran and about 80% for 40 kDa dextran. However, using higher concentrations of polyions (10 mg/ml) results in shells with higher diffusional restrictions. This is evidenced by the about 80% reduction in diffusion for a 10 kDa dextran as compared to alginate beads.

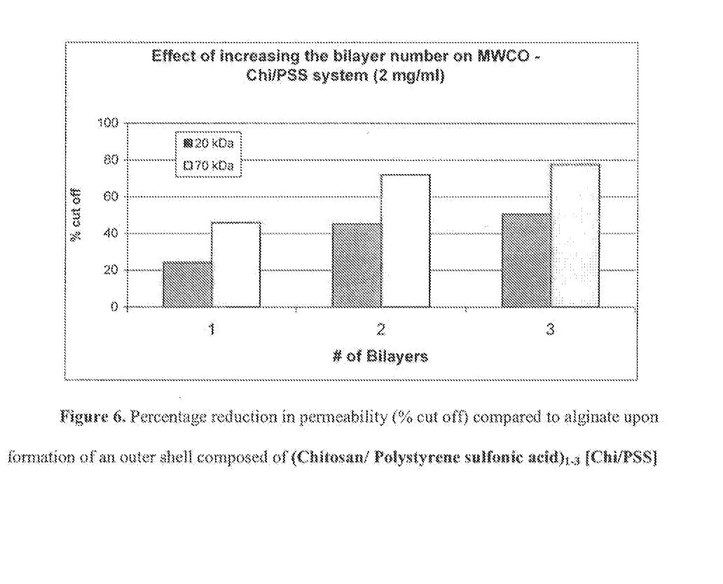

Some effects of varying the number of bilayers is shown in FIG. 6 for a Chitosan/PSS system. Increasing the number of bilayers from 1 to 3 increases the permeability of 20 kDa and 70 kDa dextran. The outer shell formed with 1 bilayer exhibits an about 24% cutoff for a 20 kDa dextran and an about 56% cutoff for a 70 kDa dextran; the formation of 3 bilayers results in higher degree of restriction, about 50% and about 78% respectively for 20 kDa and 70 kDa dextrans. As expected, in this example, the cutoff obtained for a 2 bilayer system is well within the range of 1 and 3 bilayer systems.

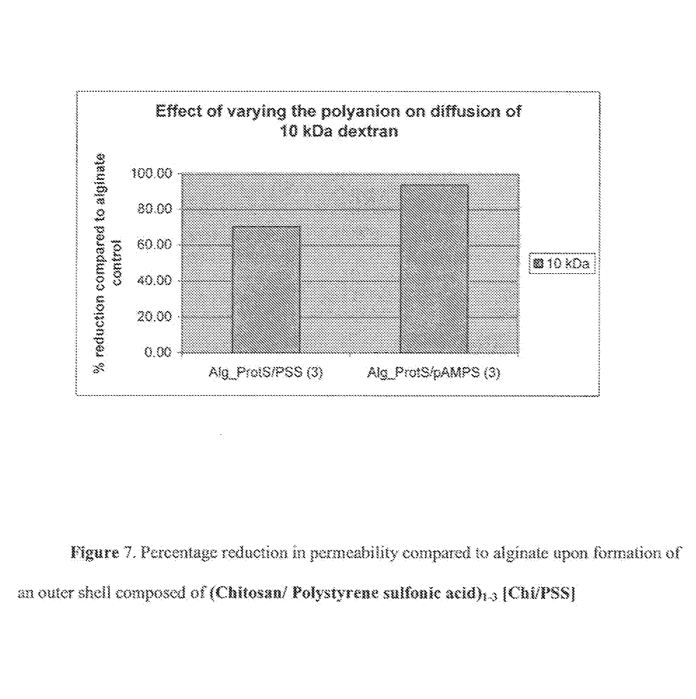

By using polyelectrolytes with different ionic strengths, as exemplified, in FIG. 7, one can produce outer shells with different degrees of diffusional restrictions. When PSS is used as the counter ion for protamine sulfate, the diffusion of MW 10 kD-40 kD FITC dextran of varies in the range of about 5% to about 35% as compared to alginate beads. Using a polyanion of higher ionic strength such as polyAMPS as the counter ion for protamine sulfate results in outer shells with much tighter porosity. The diffusion of 10 kD-40 kD dextran in these beads is less than about 10% of that in alginate beads.

In some embodiments, Alginate beads are crosslinked by divalent cations such as Ca, Ba or Sr. One can monitor a change in capsule physical integrity and/or mechanical strength by measuring its solubility in a low-calcium containing buffer, such as 0.9% NaCl, or in a Ca chelating buffer such as EDTA or sodium citrate. In vivo stability of the capsules is a function of ion exchange between the capsules and the surrounding fluid (e.g., intraperitoneal fluid). A capsule crosslinked by a divalent cation, such as calcium or barium, can be easily exchanged into a low-Ca or Ba containing medium. This results in the physical disintegration of the capsule.

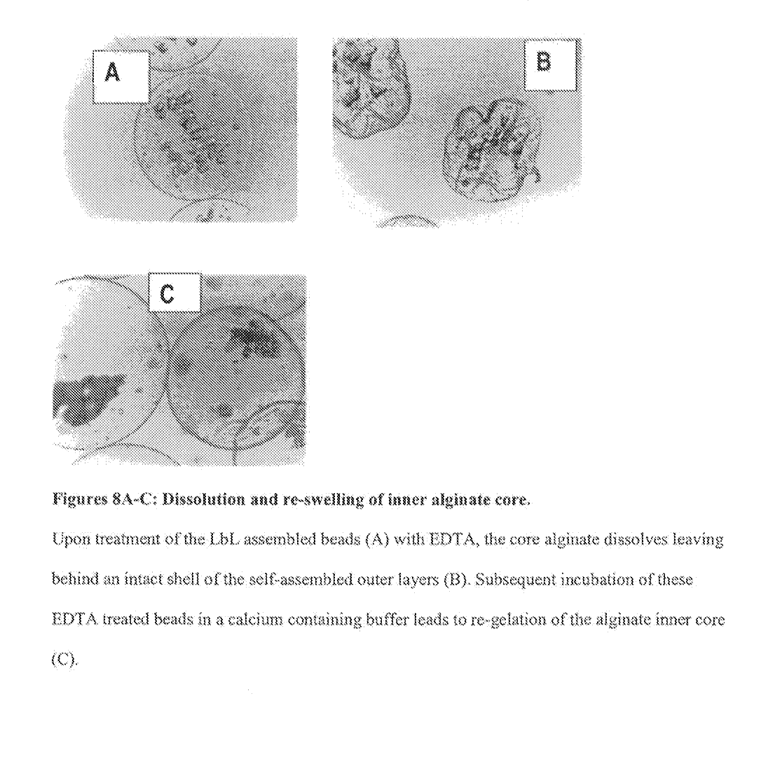

Formation of an outer shell via the molecular assembly used in some embodiments of the present invention can significantly reduce the ion exchange between capsules and surrounding fluid, and therefore, can enhance capsule stability and integrity. Solubility differences of alginate beads before and after outer shell formation is shown in FIG. 8. Alginate beads dissolve instantaneously in a calcium chelating buffer such as EDTA. In comparison, capsules of the present invention swell in EDTA as the inner alginate bead dissolves (FIG. 8A) and the outer shell remains intact. The results of treating LbL assembled beads with EDTA is shown in FIGS. 8B and 8C respectively: the core alginate dissolves, leaving the intact shell of the self-assembled outer layers intact. Subsequent incubation of the EDTA treated beads in a calcium containing buffer leads to re-gelation of the alginate inner core.

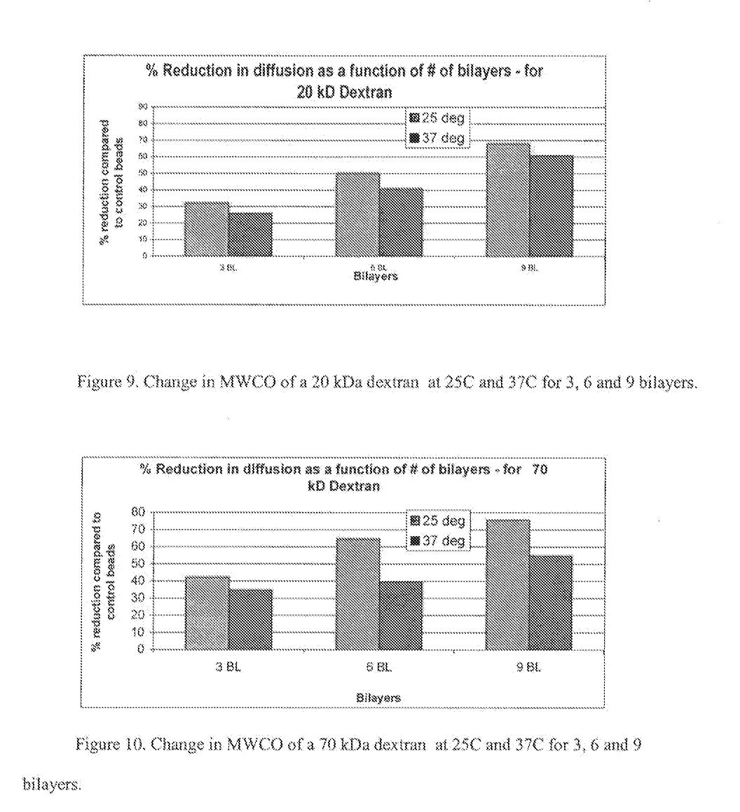

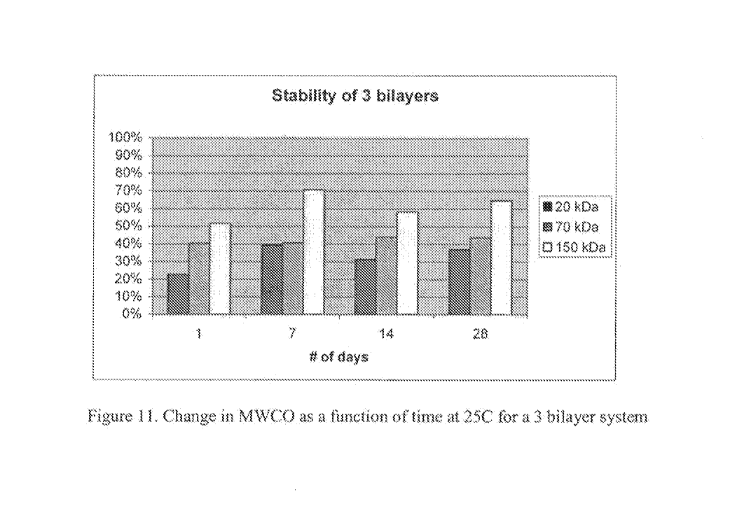

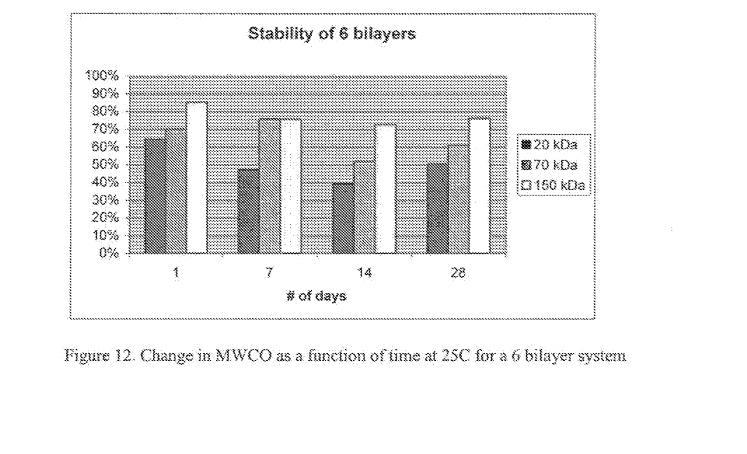

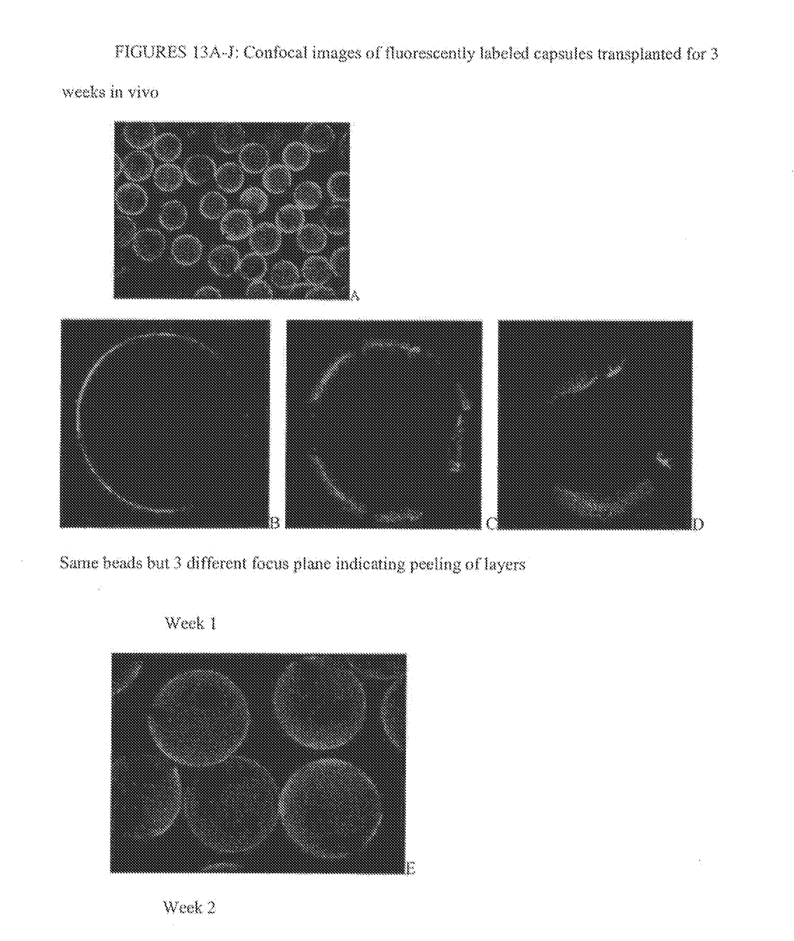

Stability of the capsules formed according to some embodiments of the present invention has been evaluated both in vitro and in vivo. In vitro evaluations were performed by culturing beads in 10% serum containing culture medium (CMRL) at different temperatures for up to approximately 30 days. Capsule stability was measured by monitoring the changes in permeability of FITC-Dextran as a function of bilayer number, time and temperature (FIGS. 9-12). As shown in FIGS. 9 and 10, the capsules exhibit stability and maintain permeability restrictions (MWCO) at both 25° C. and 37° C. The permeability restrictions are higher at 25° C. than at 37° C., because the alginate-based core beads undergo swelling at the higher temperature. Furthermore, capsules maintain their MWCO for up to about 30 days (see FIGS. 11 and 12).

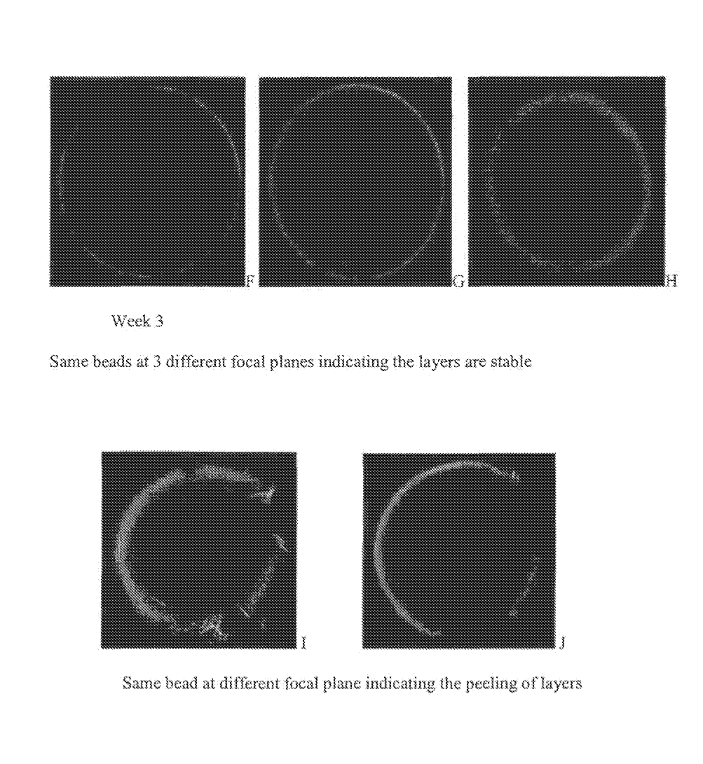

Evaluation of in vive capsule stability was performed using a fluorophore-labeled polycation. Polyallylamine-FITC was used as the inner most layer, and the outer shell was formed by a combination of Chitosan (polycation) and Bovine serum albumin (polyanion). Post transplantation, the capsules were explanted on week 1, 2, and 3, respectively. Fluorescence intensity of the explanted beads was evaluated using confocal microscopy. Lack of bead surface fluorescence uniformity can be explained by capsule instability or the peeling-off of shell layers. Typical fluorescence images of beads are shown in FIG. 13.

Capsules of the present invention are typically used to encapsulate live cells, which are usually of mammalian origin. Examples of the encapsulated cells include, without limitation, one or more of the following: pancreatic islet cells (e.g., human and/or porcine); liver cells, stem cells; neurotrophin cells; and, Fac8 cells.

The capsules further provide for maintaining the viability of the various cells (e.g., islets) post transplantation. The viability of cells on the first day is oftentimes greater than 90% or greater, but may be, for example, 75% or greater, 80% or greater, or 85% or greater. On day 5 (after about 96 hours), the viability is typically 75% or greater, preferably 80% or greater, and more preferably 85% or greater. On day 10 (after about 216 hours), the viability is oftentimes 65% or greater and may be 75% or greater.

Glucose-stimulated insulin secretion from β-cells is important with respect to maintaining glucose homeostasis. Signals that stimulate insulin release are derived from the intracellular metabolism of glucose, rather than from a ligand—receptor interaction. This process triggers an acceleration of β-cell metabolism, which ultimately leads to insulin exocytosis. Increases in oxygen consumption upon glucose stimulation in islets provides direct evidence for an accelerated rate of β-cell metabolism that accompanies increases in insulin secretion. Together, these observations suggest that detection of the islet oxygen consumption rate (OCR) in response to glucose may provide an in vitro means to rapidly and robustly assess the functional viability of an islet preparation prior to transplantation.

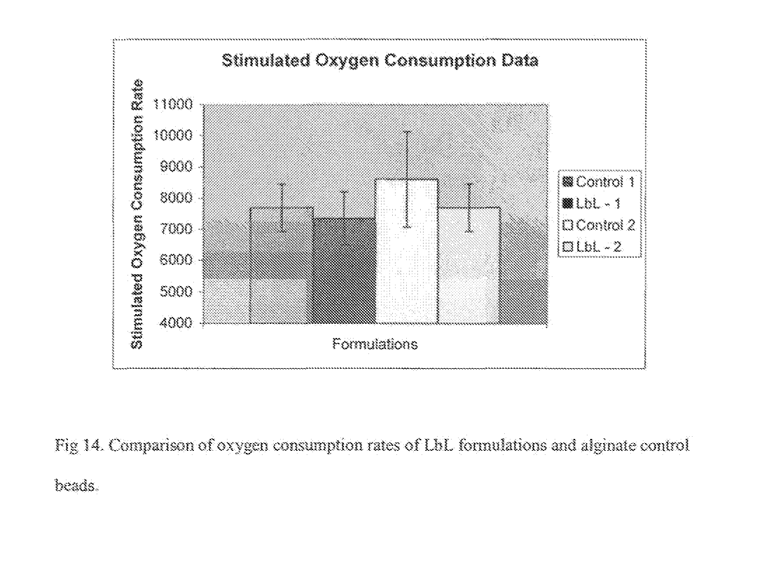

OCR for formulations of the present invention is similar to that of formulations of alginate-based capsules containing the same type and number of cells. Typical results for two formulations, in comparison to alginate controls, are shown in FIG. 14. Typically, the OCR is within about 25 percent of that of alginate-based capsules (i.e., capsules where the capsule membrane is only composed of alginate-based polymers). In certain cases, it is within about 15 percent or even about 10 percent of similarly situated alginate-based capsules. These data show that once the islets or different cell lines are encapsulated in the 3 dimensional structure (alginate capsules), the process of creating the outer shell using the multilayer approach will have no impact on the health and functioning of encapsulated cells.

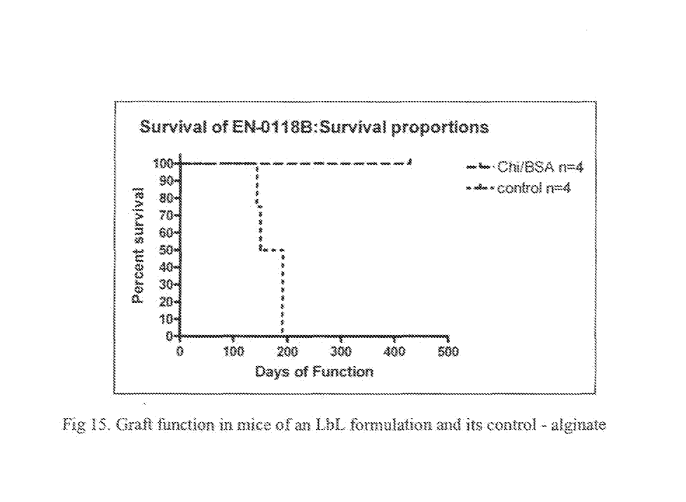

Capsules of the present invention exhibit enhanced biocompatibility as compared to alginate-based capsules. Graft function in mice of cells encapsulated within capsules of the present invention (e.g, Chi/BSA)₃ typically lasts at least about twenty-five percent longer than that of cells encapsulated within alginate-based capsules. Oftentimes, graft function lasts at least about fifty percent, about seventy-five percent, or even about one hundred percent longer than that of cells encapsulated within alginate-based capsules. Typical results of graft function for an LbL formulation and the alginate control are shown in FIG. 15.

Graft function of transplanted cells using capsules of the present invention typically results in a graft survival rate of cells of greater than about ninety-five percent after one hundred days. Oftentimes, the graft survival rate of cells is greater than about ninety-five percent after about 150, about 200, about 250, about 300, or even about 350 days.

Encapsulated islet cells produced according to the present invention may be transplanted into subjects as a treatment for a variety of diseases (e.g., islets for insulin-dependent diabetes); such transplantation may be into the peritoneal cavity, or other suitable location, within the subject. Where diabetes is the targeted disease, an amount of encapsulated islet cells to produce sufficient insulin to control glycemia in the subject is provided by any suitable means, including, but not limited to, surgical implantation and intraperitoneal injection. The International Islet Transplant Registry has recommended transplants of at least 6,000 islets, equivalent to 150 μm in size, per kilogram of recipient body weight, to achieve normoglycemia. However, it will be apparent to those skilled in the art that the quantity of capsules transplanted depends on the ability of the capsules to provide insulin in vivo, in response to glucose stimulation. One skilled in the art will be able to determine suitable transplantation quantities of capsules, using techniques as are known in the art.

The following are various, specific capsules of the present invention that are meant to exemplify, rather than limit, the present invention:

Composition 1
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—pancreatic islet cells
Effective Pore Size—less than 50 nm

Composition 2
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—two
Material Included in Capsule—pancreatic islet cells
Effective Pore Size—less than 50 nm

Composition 3
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—three
Material Included in Capsule—pancreatic islet cells
Effective Pore Size—less than 50 nm

Composition 4
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—four
Material Included in Capsule—pancreatic islet cells
Effective Pore Size—less than 50 nm

Composition 5
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—five
Material Included in Capsule—pancreatic islet cells
Effective Pore Size—less than 50 nm

Composition 6
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—at least one
Material Included in Capsule—pancreatic islet cells
Effective Pore Size—less than 10 nm

Composition 7
Base Membranous Structure—alginate
Positive Polyelectrolyte—chitosan modified with poly(ethylene glycol)
Negative Polyelectrolyte—poly(styrene sulfonate)
Ordering of Polyelectrolytes—chitosan before poly(styrene sulfonate)
Number of Bilayers—at least one
Material Included in Capsule—pancreatic islet cells
Effective Pore Size—less than 50 nm

Composition 8
Base Membranous Structure—alginate
Positive Polyelectrolyte—chitosan modified with an RGD motif
Negative Polyelectrolyte—poly(styrene sulfonate)
Ordering of Polyelectrolytes—chitosan before poly(styrene sulfonate)
Number of Bilayers—at least one
Material Included in Capsule—pancreatic islet cells
Effective Pore Size—less than 50 nm

Composition 9
Base Membranous Structure—alginate
Positive Polyelectrolyte—chitosan modified with an RGD motif
Negative Polyelectrolyte—poly(styrene sulfonate)
Ordering of Polyelectrolytes—chitosan before poly(styrene sulfonate)
Number of Bilayers—at least one
Material Included in Capsule—pancreatic islet cells
Effective Pore Size—less than 50 nm

Composition 10
Base Membranous Structure—alginate
Positive Polyelectrolyte—poly(L-lysine)
Negative Polyelectrolyte—poly(styrene sulfonate)
Ordering of Polyelectrolytes—poly(L-lysine) before poly(styrene sulfonate)
Number of Bilayers—at least one
Material Included in Capsule—pancreatic islet cells
Effective Pore Size—less than 50 nm

Composition 11
Base Membranous Structure—alginate
Positive Polyelectrolyte—poly(allylamine hydrochloride)
Negative Polyelectrolyte—poly(styrene sulfonate)
Ordering of Polyelectrolytes—poly(allylamine hydrochloride) before poly(styrene sulfonate)
Number of Bilayers—at least one
Material Included in Capsule—pancreatic islet cells
Effective Pore Size—less than 50 nm

Composition 12
Base Membranous Structure—alginate
Positive Polyelectrolyte—poly(ethylene imine)
Negative Polyelectrolyte—poly(styrene sulfonate)
Ordering of Polyelectrolytes—poly(ethylene imine) before poly(styrene sulfonate)
Number of Bilayers—at least one
Material Included in Capsule—pancreatic islet cells

Composition 13
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—neurotrophin cells
Effective Pore Size—less than 50 nm

Composition 14
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—Fac8 cells
Effective Pore Size—less than 50 nm

Composition 15
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—pancreatic islet cells
Effective Pore Size—less than 50 nm
Cell Viability—At least 90% on the first day post transplantation

Composition 16
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—pancreatic islet cells
Effective Pore Size—less than 50 nm
Cell Viability—At least 85% on the fifth day post transplantation

Composition 17
Base Membranous Structure—alginate
Positive Polyelectrolyte—chitosan
Negative Polyelectrolyte—poly(styrene sulfonate)
Ordering of Polyelectrolytes—chitosan before poly(styrene sulfonate)
Number of Bilayers—one
Material Included in Capsule—pancreatic islet cells
Effective Pore Size—less than 50 nm
Cell Viability—At least 75% on the tenth day post transplantation

More examples of compositions that are biocompatible and provide MWCO necessary for long term graft function are shown in Table 1 below.

TABLE 1
Typical formulations of polyelectrolyte based capsules (LbL)

| | | | MWCO | |
|---|---|---|---|---|
| I.D. | Inner core | Composition of outer shell | 10 k | 40 k |
| 1 | Alginate | (HDM_PSS 0.2/2.0)₃ | −30 | −47 |
| 2 | Alginate | (HDM_PSS 0.2/2.0)₃ + PEG | −18 | −65 |
| 3 | Alginate | (HDM/PSS 0.2/2)₁₋₃ | 16 | −33 |
| 4 | Alginate | (HDM_PSS 0.2/2.0)₂ + | −16 | −12 |
| | | (PROT S/PSS .2/.2) | | |
| 5 | Alginate | (HDM_PSS 0.2/2.0)₃ + | −31 | −35 |
| | | (PROT S/PSS .2/.2) + PEG | | |
| 6 | Alginate | (HDM/PAMPS_.2/2)₃ | −5 | −43 |
| 7 | Alginate | (HDM/PAMPS_.2/2)₃ + PEG | 9 | −15 |
| 8 | Alginate | (HDM/PAMPS 0.2/2)₂ + | −6 | −19 |
| | | (ProtS/PAMP 0.2/2)₂ + PEG | | |
| 9 | Alginate -g- | (HDM/PAMPS_.2/2)₃ | −25 | −11 |
| | pAMPS | | | |
| 10 | Alginate -g- | (HDM/PAMPS_.2/2)₃ + PEG | −13 | −25 |
| | pAMPS | | | |
| 11 | Alginate -g- | (HDM/PAMPS_.2/2)₂ + | 7 | −21 |
| | pAMPS | (ProtS/PAMP 0.2/2)₂ + PEG | | |
| 12 | | | | |
| 13 | Alginate | (ProtS/PSS 0.2/.2)₁ + PEG | −7 | −24 |
| 14 | Alginate | (ProtS_PSS 0.2/.2)₂ + PEG | 62 | 102 |
| 15 | Alginate | (ProtS_PSS 0.2/.2)₃ | 32 | 81 |
| 16 | Alginate | (ProtS_PSS 0.2/.2)₃ + PEG | 71 | 106 |
| 17 | Alginate | (ProtS_PSS 0.2/.2)₃ | 83 | 77 |
| 18 | Alginate | (ProtS_PSS 0.2/.2)₃ + PEG | 52 | 95 |
| 19 | Islet | (ProtS_PSS 0.2/.2)₃ + PEG | 70 | 77 |
| | encapsulated | | | |
| | Alginate | | | |
| 20 | Alginate | (ProtS/PSS 0.2/.2)₁₋₃ | 49 | 75 |
| 21 | Alginate | (ProtS_PSS 0.2/2)₃ | −18 | −40 |
| 22 | Alginate | (ProtS_PSS 0.2/2)₃ + PEG | 6 | −10 |
| 23 | Alginate | (ProtS_PAMPS 0.2/.2)₃ + PEG | 10 | 38 |
| 24 | Alginate | (ProtS/PAMP 0.2/.2)₃ + PEG | 20 | 67 |
| 25 | Alginate | (PROT.S/PAMPS_.2/.2)3 | 79 | 105 |
| 26 | Alginate | (PROT.S/PAMPS_.2/.2)3 + PEG | 94 | 98 |
| 27 | Alginate -g- | (ProtS/PAMPS 0.2/.2)2 + PEG | 33 | 2 |
| | pAMPS | | | |
| 28 | Alginate -g- | (PROT.S/PAMPS_.2/2)3 | 48 | 69 |
| | pAMPS | | | |
| 29 | Alginate -g- | (PROT.S/PAMPS_.2/2)3 + PEG | 43 | 72 |
| | pAMPS | | | |

Abbreviations:
HDM—Hexamethirine dibromide;
PSS—Polystyrene sulfonic acid;
ProtS—protamine sulfate;
PAMPS—poly(acrylamidomethyl propane sulfonic acid);
PEG—Polyethylene glycol.

### EXAMPLES

### Example I

### General Synthesis of Modified Alginates

Polymer grafted alginate was prepared by a radical solution polymerization of acrylated monomers in the presence of alginate and an aqueous free radical initiator. Sodium alginate and at least one type of acrylated monomer were dissolved in HEPES buffered saline to yield a solution of 3% (w/w) alginate and a final monomer concentration of 0.04 to 5.0 mmol. Aqueous initiator was added to the alginate/monomer solution in an amount of 0.5 wt % of total monomer. The entire mixture was vortexed and allowed to react for 4 hr with additional vortexing at 15 min intervals.

### Example 2

### Capsule Fabrication

Islets were suspended in 2.0% of alginate or modified alginate and placed in a droplet generator adapted from that of Walters et al., J. Appl. Biomater. 3:281 (1992). Droplets generated from islets suspended in the alginate or modified alginate solution were collected in a funnel containing 1.1% CaCl₂, where they gelled.

### Example 3

### Polyelectrolyte Deposition by Sequential Layering

### LbL Process

Alginate beads, bearing net negative surface charge density are incubated with a polycation solution in a medium containing 1.8 mM CaCl2 for 5 minutes at room temperature, with rocking. After 5 min, the beads are allowed to settle and the supernatant is removed using a pipette. The beads are rinsed three times with serum free culture medium. They are subsequently incubated with a solution of the polyanion for min. with rocking. The supernatant is removed and the beads are rinsed three times with serum free culture medium. The process of incubation in polycation, washing, incubation in polyanion and washing result in the formation of a single bilayer. This process is repeated to increase the number of bilayers as desired for a particular application. The washed beads are further cultured at 25 C in a serum free culture medium.

### Example 4

### Graft Function in Mice

Islets encapsulated in capsules composed of (Chi/BSA)3 were transplanted into mice made diabetic by a single IP injection of streptazotozin. Islet dose for each experiment ranged from 1.5 k to at a dose of 2.5 k IEQ/mouse.

It is seen that compositions and methods are provided. One skilled in the art will appreciate that the present invention can be practiced by other than the various embodiments, which are presented in this description for purposes of illustration and not of limitation, and the present invention is limited only by the claims that follow. It is noted that equivalents for the particular embodiments discussed in this description may practice the invention as well.

While various embodiments of the present invention have been described above, it should be understood that they have been presented by way of example only, and not of limitation. Moreover, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Additionally, with regard to operational descriptions and method claims, the order in which the steps are presented herein shall not mandate that various embodiments be implemented to perform the recited functionality in the same order unless the context dictates otherwise.

The claimed invention is:

1. A composition comprising a plurality of capsules, wherein each capsule comprises:
an inner core coated with an alginate and comprising at least one porcine islet cell; and
an outer shell enclosing the inner core and comprising at least one positive polyelectrolyte and at least one negative polyelectrolyte arranged in oppositely charged layers.

2. The composition of claim 1, wherein the alginate is a polymer-modified alginate.

3. The composition of claim 1, wherein the positive polyelectrolyte is selected from one or more of the group consisting of: chitosan, protamine sulfate, polybrene, poly-(L-lysine), poly(allylamine hydrochloride), poly(ethylene imine) and poly(ethylene glycol-co-dimethylaminoethyl methacrylate); and the negative polyelectrolyte is selected from one or more of the group consisting of: poly(styrene sulfonate), polyacrylamideomethyl propane sulfonic acid, poly(lactic acid), cellulose sulfate, alginate, hyaluronic acid, chondroitin sulfate and poly(ethylene glycol-co-methacrylic acid).

4. The composition of claim 1, wherein a positive polyelectrolyte is first disposed between the inner core and a negative polyelectrolyte in the outer shell.

5. The composition of claim 1, wherein a negative polyelectrolyte is first disposed between the inner core and a positive polyelectrolyte in the outer shell.

6. The composition of claim 1, wherein the polyelectrolyte disposed in the outermost portion of the outer shell comprises a negative polyelectrolyte modified with a protein and polyethylene glycol.

7. The composition of claim 1, wherein the polyelectrolyte disposed in the outermost portion of the outer shell comprises a negative polyelectrolyte modified with at least one anticytokine antibody or at least on RGD motif.

8. The composition of claim 1, wherein the capsules exhibit a porosity control equal to the diffusional restriction of dextrans of defined molecular weight and wherein the diffusional restriction is controlled in the range of about twenty percent molecular weight cutoff to about ninety percent molecular weight cutoff for a 10 kD dextran.

9. The composition of claim 1, wherein the capsules exhibit a porosity control equal to the diffusional restriction of dextrans of defined molecular weight, and wherein the diffusional restriction is controlled in the range of about thirty percent molecular weight cutoff to about eighty percent molecular weight cutoff for a 10 kD dextran.

10. The composition of claim 1, wherein the capsules exhibit a porosity control equal to the diffusional restriction of dextrans of defined molecular weight, and wherein the diffusional restriction is controlled in the range of about thirty percent molecular weight cutoff to about ninety percent molecular weight cutoff for a 40 kD dextran.

11. The composition of claim 8, wherein the capsule comprising an effective pore size of less than about 10 nm.

12. The composition of claim 1, further comprising at least one anti-inflammatory drug conjugated to the outer shell, at least one immuno-suppressive drug conjugated to the outer shell or at least one targeting-type molecule conjugated to the outer shell.

13. The composition of claim 1, further comprising anti-apoptotic agents in the inner core.

14. A method of treating diabetes comprising the step of administering to a patient in need thereof a composition of claim 1.

15. The method according to claim 14, wherein the composition is administered through intraperitoneal injection.

16. The method according to claim 14, wherein the cells in the composition exhibit a viability of greater than about 80 percent within 24 hours after administration.

17. The method according to claim 16, wherein the cells in the composition exhibit a viability of greater than about 80 percent within 96 hours after administration.

18. A method for the formation of a polyelectrolyte-based capsule for porcine islet cell encapsulation comprising the steps of:
a) suspending porcine islet cells in an alginate solution to form a suspension;
b) generating droplets of the suspension;
c) gelling the droplets to form cell-encapsulated alginate beads;
d) incubating the alginate beads in a first polyelectrolyte solution under conditions to form a first polyelectrolyte layer on the alginate beads;
e) rinsing the alginate beads having a first polyelectrolyte layer;
f) incubating the alginate beads having a first polyelectrolyte layer in a second polyelectrolyte solution under conditions to form a second polyelectrolyte layer on the first electrolyte layer resulting in alginate beads encapsulated within a polyelectrolyte bilayer, wherein the second polyelectrolyte has the opposite charge from the first polyelectrolyte;
g) rinsing the encapsulated alginate beads; and
h) optionally repeating steps d), e), f) and g) to form additional polyelectrolyte bilayers on the encapsulated alginate beads;
wherein neither the first polyelectrolyte nor the second polyelectrolyte is alginate.

19. The method of claim 18, wherein the alginate is a polymer-modified alginate.

20. The method of claim 18, further comprising step i), conjugating the outer shell of the first capsule to at least one anti-inflammatory drug, at least one immuno-suppressive drug or at least one targeting-type molecule.

21. The method of claim 18, further comprising the step of modifying the inner core with a cell adhesive protein moiety.

22. The method of claim 18, further comprising in step a) the step of adding anti-apoptotic agents that are encapsulated in the inner core.

23. The method of claim 18, wherein the first polyelectrolyte is a positively charged polyelectrolyte selected from a group consisting of chitosan, protamine sulfate, polybrene, poly(L-lysine), poly(allylaminehydrochloride), poly(ethylene imine) and poly(ethylene glycol-co-dimethylaminoethyl methacrylate); or wherein the first polyelectrolyte is a negatively charged polyelectrolyte selected from a group consisting of poly(styrene sulfate), polyacrylamideomethyl propane sulfonic acid, poly(lactic acid), cellulose sulfate, hyaluronic acid, chondroitin sulfate and poly(ethylene glycol-co-methacrylic acid).

24. A porcine islet cell capsule comprising an inner core coated with an alginate and comprising at least one porcine islet cell; and an outer shell enclosing the inner core and comprising at least one positive polyelectrolyte and at least one negative polyelectrolyte arranged in oppositely charged layers.
